(19) United States
(12) Patent Application Publication
(10) Pub. No.: US 2009/0145188 A1
(43) Pub. Date: Jun. 11, 2009

(54) Apparatus and methods for generating compost

(75) Inventor: Emmanuel G. Moya, Hamilton (CA)

Correspondence Address: MARK D MILLER;KIMBLE, MACMICHAEL & UPTON, 5260 NORTH PALM AVENUE, SUITE 221, FRESNO, CA, 93704, US

(73) Assignee: Halton Recycling Limited

(21) Appl. No.: 11/999,917

(22) Filed: Dec. 7, 2007

(51) Int. Cl. C05F 17/00; C12M 1/00

(52) U.S. Cl. 71/9; 435/290.4

(57) ABSTRACT

The present invention provides apparatus and methods for shortening the time needed to convert mixed pulp into unlimited use and relatively odor-free compost down from several weeks to several days, and for producing the compost without the need for a large or remote outdoor area. In addition, the process may be modified so as to improve the quality of the compost so that it is made more suitable for particular soil applications.

### BACKGROUND OF THE INVENTION

1. Field of the Invention

The present invention relates to the generation of compost from kitchen, yard and other waste, and more particularly to apparatus and methods for accelerating the generation of compost that is relatively odor-free and that meets certain pre-determined soil amendment requirements.

2. Description of the Prior Art

In the process of composting, microorganisms break down organic matter and eventually produce a relatively stable organic end product. Source material for composting may come from a variety or sources, among these are yard waste (cuttings, clippings, manure, etc.) and unconsumed or inedible food. Composting typically goes through three phases: (1) a mesophilic or moderate-temperature phase, (2) a thermophilic or high-temperature phase, and (3) a lengthy cooling and maturation phase.

It is common to separate kitchen waste products (food, peelings, etc.) from other waste (metals, aluminum, plastic, etc.) with the resulting organic liquid being fed into an anaerobic (mesophilic) digester for the first phase of composting. This phase may last for approximately 1-3 days. The result of this digestion is the production of methane gas (which may be used to produce electricity in a co-generator) and a semi-solid material. This material is further processed (phase 2) for several more days in an aerobic (thermophilic) composting unit at a temperature of between 55° C.-77° C. Vertical Composting Units (VCUs), such as those made by VCU New Zealand, are used for this phase and perform the thermophilic digestion. In this process, the pH of the soil is neutralized, a moisture content is developed, bacteria is eradicated (especially pathogenic bacteria), and the material is stabilized.

The pre-compost pulp material produced by VCUs generally has a powerful odor and a high moisture content. In addition, because of their vertical orientation, material must be fed into the top of a VCU. This can cause at least two problems. First, the compacting weight of the product has a tendency to become anaerobic (not aerobic) at the lower stages before removal, trapping moisture, and causing air to be channeled, not circulated. Second, the introduction of new feed from the top contributes to the regeneration of new contaminants within the cell where the compost product is being matured. The result of these combined effects is that more odor is produced, pathogens are not eradicated, and the moisture content is higher.

To reduce the odor and moisture of such pre-compost pulp, a bulking agent such as wood chips, saw dust or other similar material may be mixed with the pre-compost pulp at a pre-determined ratio to form a mixed pulp. Additional ingredients may also be added at this time in order to produce compost having desired final characteristics and carbon-nitrogen (C:N) ratios for use on particular applications and/or to conform to government regulations. These ingredients include without limitation pH conditioners such as lime, “bloodmeal” for Nitrogen, etc. This pre-compost pulp material has traditionally been converted into compost using outdoor windrow composting. The windrow composting process (phase 3) calls for turning over the pulp/compost on a daily basis, and typically takes 6-8 weeks from VCU post maturation to convert the mixed pulp into compost. This process requires considerable outdoor space that must be away from populated areas because of the odorous steam.

It is therefore desirable to reduce the time needed to produce usable odor-free compost, to reduce the outdoor the space necessary for such composting, and to make it possible to provide such composting near populated areas.

### SUMMARY OF THE INVENTION

The present invention provides apparatus and methods for shortening the time needed to convert mixed pulp into usable and relatively odor-free compost down from several weeks to several days, and for producing the compost without the need for a large or remote outdoor area. In addition, the process may be modified so as to improve the quality of the compost so that it is made more suitable for particular soil applications.

The present invention is designed primarily to receive and process pulp that has been processed in a VCU, typically for around seven days. This pre-matured mix is then diverted to an open trommel or screen. In this step, the mixed product is further classified, with items larger than approximately two inches in diameter being separated and removed. During this process, the remaining product is gathers oxygen naturally. The classified mixed pre-matured compost (smaller than approximately 2″ in diameter) is then diverted to a maturation tank for a number of days, after which the pulp may then be ready, or it may be transferred to a second large tank and further processed for another number of days. The processing times in each of these tanks may vary from one to six days each, or more, with each processing time averaging approximately three days (for a combined average total of approximately six days). If a single tank is used, the processing time will be closer to six days; if dual tanks are used, the processing time in each tank will be closer to three days. At the end of this (dual) processing, the material is in the form of relatively odor-free compost/humus that can be bagged or shipped.

In alternative embodiments, the invention may receive and process mixed product (organic pulp) directly after it has been blended with woodchips or the like, without having been pre-processed in a VCU. In these embodiments where the VCU is bypassed (e.g. for reasons such as repair or bottleneck), the product may be processed in the maturation tank(s) of the present invention for approximately 12-14 days.

In some embodiments, the apparatus of the invention includes a large tank that may have a horizontal or vertical (silo) orientation, referred to as a VCMT (in-vessel compost maturation tank). In the preferred embodiments, the tank has a generally horizontal orientation and is provided in a generally cylindrical form. Such a tank may be, for example and without limitation, approximately 50 feet long having a 12½ foot diameter, although a tank of any suitably size and shape may be used. A rotatable hollow shaft is provided alone the inside of the tank having two sets of augers mounted on it. Such a shaft may, for example and without limitation, have a diameter of about 12 inches. A larger auger causes the material to travel in one direction, and the smaller auger causes it to travel in the opposite direction—resulting in a mixing effect. For example, and without limitation, a diameter for the larger auger may be in the range of about sixty to about seventy inches (60″-70″), and a diameter for the smaller auger may be about forty-eight inches (48″), or in the range of about forty-two to about fifty-four inches (42″-54″). In some embodiments, a set of flexible spring loaded tines are provided along the shaft. Paddles or scrapers may be provided at the ends of these tines. These tines should be long enough to stir the annular space between the edge of the larger auger and the inside edge of the tank, to reach the compost beyond the augers and ensure that internal dead space in the tank is minimized.

In these embodiments, the auger shaft is hollow and is provided with a set of air openings or nozzles that are spaced apart along some or all of the auger shaft. For example and without limitation, in some embodiments, a perforated hollow pipe (sch. 80 pipe) may be used. A pipe, hose or other input provides forced air containing oxygen into the auger shaft which exits through the openings/nozzles into the tank. In preferred embodiments, the air openings/nozzles are uniformly spaced apart, and installed at 90 degree increments from each other in a semi-helical fashion. A pressurized air source for providing heated air causes an incoming air stream to be constantly introduced through the auger pipe. The introduction of heated air helps minimize leachates that may result from condensation. In preferred embodiments, a multi-directional aeration disbursement device is provided at each opening to provide better air distribution into the compost pulp mix in the tank, and to prevent clogging.

In the vertical embodiments of the invention, warm air is also forced into a peripheral space along the inside wall of the tank. This space is formed using an interior wall that is adjacent to the inside wall of the tank forming a plenum. The interior wall is provided with openings which allow air to exit from the plenum into the tank.

In most embodiments, the internal temperature of the tank is maintained between approximately 45° C. and 65° C. which may be accomplished using the introduction of heated air, and insulation provided around some or all of the tank. For example, insulation may be provided on the top and sides, and/or a heating jacket may be provided from side to the bottom. The tank is vented on the top, and may include a vacuum-type blower for positive evacuation of moisture and spent odorous air. This spent air should be sent to a main biofilter for treatment before releasing to the environment. This air may be treated with oxygen (so as to reach an air-to-oxygen ratio of approximately 20:1) and re-introduced into the mixture in the tank. The maturation tank has an inlet feed opening for receiving the pre-matured compost material, and a controlled outlet for the matured compost material. In the horizontal embodiments, the inlet should be located at or near the top of the tank; in the vertical embodiments, the inlet should be located near but above the bottom of the tank.

In some embodiments, ultraviolet lighting may be provided in the top of the tank. For example, three sets of high intensity ultraviolet lights may be provided in spaced relationship along the inside of the top of the tank. This ultraviolet light eliminates additional unwanted pathogens while the product is being mixed.

In horizontal embodiments, a plurality of openings or spray nozzles may be provided along the inside of the top of the tank. These openings are used to introduce various fluids into the material, depending upon the desired output content. The openings may be provided in staggered, uniform or random locations inside the tank. Such openings may be provided on a pipe extending along some or all of the length of the inside of the tank. Multiple pipes with their own sets of openings may be provided to allow introduction of different materials from different sources. Such a pipe may, for example and without limitation, have a diameter of about one inch, with openings or nozzles spaced about 12-inches apart. In vertical embodiments, a single opening or nozzle is provided immediately adjacent to the lower input for pre-compost pulp material allowing the fluid introduced to be mixed with the material as it enters the tank.

Normally, certain enzymes or microorganisms are introduced through these openings or nozzles in order to achieve certain levels of nutrients (e.g., nitrogen, phosphorous, potassium, etc.), and/or fungi in the final humus product. The purpose of introducing a certain type and/or mixture of enzymes or microorganisms is to increase the fungal colony in the resulting matured compost for purposes such as minimizing if not completely eliminating the use of pesticides or insecticide in the farm soil where the compost is applied. The inoculation/spray of particular enzymes or microorganisms are intended to meet desired soil requirements of a particular end-product. Different requirements are desirable for compost to be used for different purposes such as grapevines, root crops, garden vegetables and/or injecting soil nutrients to replace depleted nutrients in any type of inactive soil (e.g. desert/arid lands, recovered oil sands, soil that has recently lost its nutrients from an aggressive root crop-like ginseng).

In addition, in some embodiments, in order to ensure that the compost guidelines and the governmental requirements are met, temperature sensors, level sensors, humidity sensors, pH sensors and/or sampling points (including observation and inspection windows) may also be provided in the tank, for continuous monitoring and testing of the material inside. These devices allow constant readings of basic test parameters to be taken to monitor such things as C:N ratio, oxygen uptake, and the like. Germination test and Solvita test procedures may be done on site once samples are secured, and results may be obtained within a day or two.

In use, pre-matured compost pulp from a VCU enters the compost maturation tank of the present invention through an inlet/feed. The auger-mixer moves and mixes the material in the tank, with the tines and paddles/scrapers (if provided) providing further mixing throughout the tank. A typical shaft speed may be approximately five (5) rpm, although higher and lower speeds (e.g., 10-15 rpm) may also be used depending on the consistency of the pulp material. In the horizontal embodiments, it is important that the pulp mixture cover a sufficient portion of the bottom of the tank so that liquid(s) introduced through the upper openings/nozzles do not simply drain back out of the tank. Thus, at some point, the pulp will reach a minimum level in the tank where it covers the bottom and is capable of absorbing the liquid to be introduced. At or after that point, the introduction of enzyme(s) and/or microorganisms and/or other materials may begin through the upper openings/nozzles. In some cases this may begin at, for example and without limitation, approximately ⅓ to ½ of the volume of the tank (i.e., when the level is at or near the top of the auger).

The quantity of enzyme(s) and/or microorganisms and/or other materials introduced should normally not exceed about one percent (1%) of the volume of the pulp material. The tank should not be filled more than about ¾ full by volume in order to provide sufficient space for air and the expansion of the warm pulp product. The introduction of pressurized air into the mixture through the nozzles/openings along the auger shaft also begins upon introduction of pulp into the tank. This air should be warm and contain oxygen, and should preferably be maintained at around 50-60° C. This helps keep the temperature of the compost mixture in the same range, around an average of 55° C. For example and without limitation, a uniform and steady average air velocity at about 10 feet per second (fps) per opening/nozzle at about 50-60 psig, may ordinarily be attained through all nozzles/openings with respect to both air direction (horizontal, vertical, diagonal, rotational) and magnitude (i.e. the multi directional contact of air to the product becomes more aerobic accelerating the process).

With the introduction of air through the shaft openings/nozzles, a vacuum pump or other withdrawal mechanism connected to a vent at the fop of the tank is also activated in order to begin venting the tank. This extracts the exhausted air and moisture (the air having a moisture content as condensate) from the tank. The withdrawal of air should be greater than the pressurized input of air to the hollow auger shaft to help remove moisture and particulates. A vacuum of about 103% or greater of the incoming air pressure is usually sufficient. This will ensure positive movement of spent air which is heavier than supplied air since it contains moisture and light contaminants. In addition, the spray injection of liquid supplements may be provided at different times to meet any particular soil requirements for the resulting compost. It is to be appreciated that the above steps need not necessarily be initiated simultaneously or in any particular order, but should occur at some time in conjunction with the initial introduction of pre-compost pulp material into the tank.

In the vertical embodiments, since the tank has a smaller bottom which will be readily covered by incoming pre-compost pulp product, the introduction of enzyme(s) and/or microorganisms and/or other materials at the point of product entry may begin almost immediately. In the vertical embodiments, in addition to introducing air through openings in the auger shaft, pressurized air is also introduced through openings in a peripheral plenum.

If supplied, the ultraviolet light(s) are turned on as soon as the product enters the tank in order to begin eliminating unwanted pathogens. If supplied, proximity and/or level sensors may be used to monitor the volume of the product in the tank. In addition, pH and thermo-couple sensors, monitors, acidity/alkalinity, moisture, humidity and/or temperature sensors may also be supplied at one or more points in the tank. Oxygen uptake rate sampling should preferably occur about every 24 hours. Germination tests and a C:N ratio tests should preferably occur about once daily as well. Physical measurements of C:N ratio, trace elements, pathogens, nutrients, minerals, particle size, soluble salts and maturity index (Solvita CO₂ Test) should be done by daily sampling and testing.

To achieve high levels of bacteria and pathogen eradication, a temperature range between about 45° C. and about 60° C. should be maintained for the thermophilic process. Ideally, the warm exhaust of the cogens with added amounts of oxygen (to return to at least a 20:1 air-to-oxygen ratio) is fed back into the tank through the perforated shaft to avoid having to re-heat this air. Reversing the domination of bacterial colony to fungal colony is achieved with the feeding of an enzymatic blend coupled with high intensity ultraviolet light and timed mixing at lower speeds. Longer mixing times may eventually meet the levels of protozoa and/or nematodes required by a particular soil food web.

The serration flow paths in a dynamically slow-moving compost bed using combination of multi-directional forced aeration through inverted air nozzles provides positive impact in the oxygen distribution management, moisture content reduction and temperature stability within the bed of still biologically active but pathogen-free material. The path of the bulk materials undergoes aeration and immersion through the passage of air in 3-dimensional air flow paths (vertical, horizontal and rotational) as it makes it way through an interstitial passageway and prevents channeling, accelerating the organic breakdown needed for the production of usable compost. These steps ensure the collection of possible Volatile Organic Compounds (VOC's), Sulfides and mercaptans through the condensate removal.

The incoming oxygenated warm air plus the resulting change in temperature of the compost is extracted and balanced by the vacuum at the top of the tank. The moisture and condensate are collected as well and deposited at a trap in the venting apparatus. The fugitive air collected by the vacuum contains odor causing VOC's that may be passed to a main biofilter and/or polishing carbon filters. Final compost is determined by the above measurements. Food web analysis for every harvest should be done to measure desired biomass of organisms such as dry weight, active and total bacterial count, active and total fungal counts, protozoa and nematode counts, and/or hyphal diameter. These measurements are important in determining the quality of the compost and what farm soil use it is best suited for. The resulting compost is ready for release when the C:N ratio, oxygen uptake rate and/or germination tests meet or exceed government or user-established CCME guidelines.

The final discharge of the harvested compost may be directed to a tipping floor through conveyors, where it may be screened for size requirements before bagging. Larger particles separated through the screening process may be returned to a blending area and mixed with new pulp to be processed again.

Unlimited use of the matured compost produced by the present invention should ordinarily be available within approximately six to seven (6-7) days after the pre-compost pulp mixture is introduced into the tank of the present invention from a VCU. In some cases, some compost material may be harvested every day, or every three days.

If the pulp is not pre-processed (e.g., not from a VCU) the matured compost should be available within approximately 12-14 days. In such cases, the temperature is maintained at a thermophilic level (between about 55° C. and about 75° C.) for the first approximately six to seven (6-7) days, and then reduced to a mesophilic level (between about 45° C. and about 60° C.) for the remaining approximately six to seven (6-7) days.

The resulting compost may be further screened (e.g. to ⅜″ mesh) and may be applied to a triple mix prior to bagging. The resulting product may be used in potting soil, architectural landscape, side roads improvement, household gardening, forest soil nutrients amendments, farming for vegetables, root-crops, vineyards, fruit-bearing trees, etc. The product may also be palletized to increase shelf life, for the purpose of shipping to other countries where soil nutrients are depleted and for converting arid lands into usable nutrient-rich soil.

The unique apparatus and methods of the present invention provide usable compost through the combination of the mixing/agitating action in the tank, the introduction of air, the eradication of pathogens by the UV lights while mixing, and the evacuation of spent air by balanced vacuum. In addition, the spray injection of liquid supplements may be provided to meet any particular soil requirements. These conditions accelerate the composting process, providing what nature does about 10 times faster.

It is therefore an object of the present invention to provide composting methods and apparatus for reducing the time needed to produce usable odor-free compost.

It is also an object of the present invention to provide methods and apparatus for reducing the outdoor the space necessary for composting.

It is also an object of the present invention to provide methods and apparatus to make it possible to provide relatively odor-free composting near populated areas.

It is also an object of the present invention to improve the quality of compost produced so that it is made more suitable for particular soil applications.

Additional objects of the invention will be apparent from the detailed descriptions and the claims herein.

### BRIEF DESCRIPTION OF THE DRAWINGS

FIG. 1 is a cross sectional side view of an embodiment of an in-vessel compost maturation tank of the present invention.

FIG. 2 is a cross sectional end view of the embodiment of FIG. 1.

FIG. 3 is a detailed view of an embodiment of an auger mechanism of the present invention.

FIG. 4 is a detailed view of an embodiment of an aeration system shown in FIG. 2.

FIG. 5 is a cross sectional end view of an embodiment of a tank an auger mechanism of the present invention.

FIG. 6 is a cross sectional end view of an embodiment of a tank an auger mechanism of the present invention showing compost present.

FIG. 7 is a cross sectional side view of another embodiment of an in-vessel compost maturation tank of the present invention.

FIG. 8 is a cross sectional end view of the embodiment of FIG. 6.

FIG. 9 is an end view of the embodiment of FIG. 6.

FIG. 10 is a cross sectional side view of another embodiment of an in-vessel compost maturation tank of the present invention.

FIG. 11 is an embodiment of a layout for a typical vertical composting unit area.

FIG. 12 is a schematic diagram showing an exemplary implementation of an embodiment of the present invention in a composting system.

FIG. 13 is a diagram of a prior art in-tunnel composting process.

FIG. 14 is a diagram of another prior art windrow composting process.

FIG. 15 is a diagram showing the screening and separation of different sizes of the final compost product.

### DETAILED DESCRIPTION

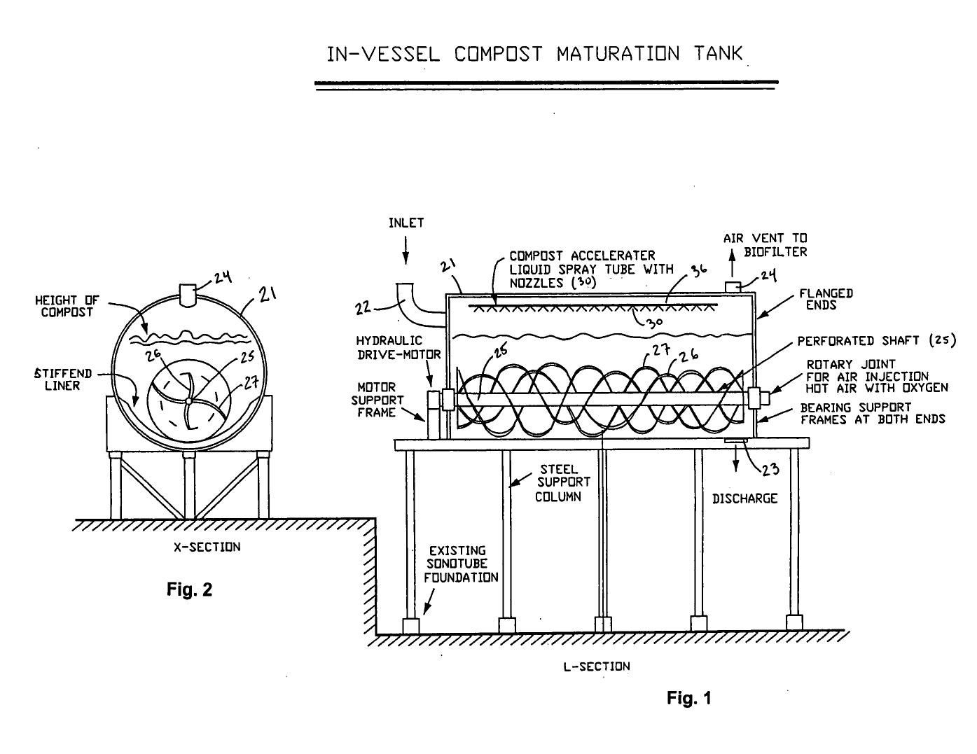

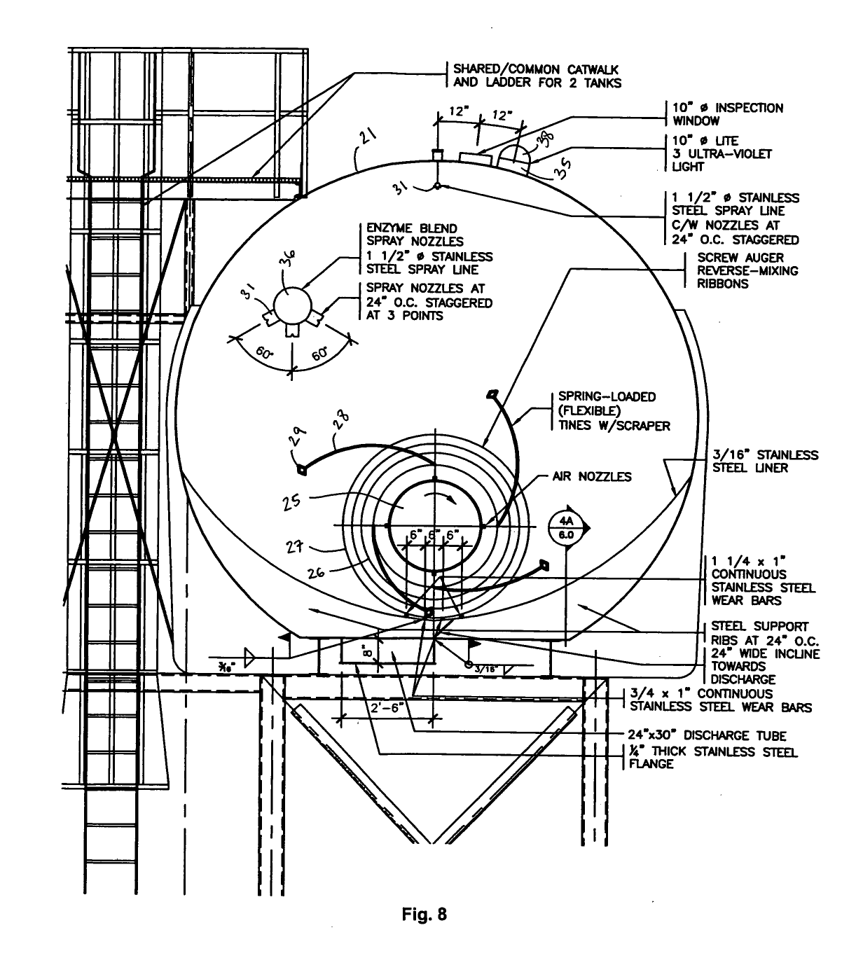

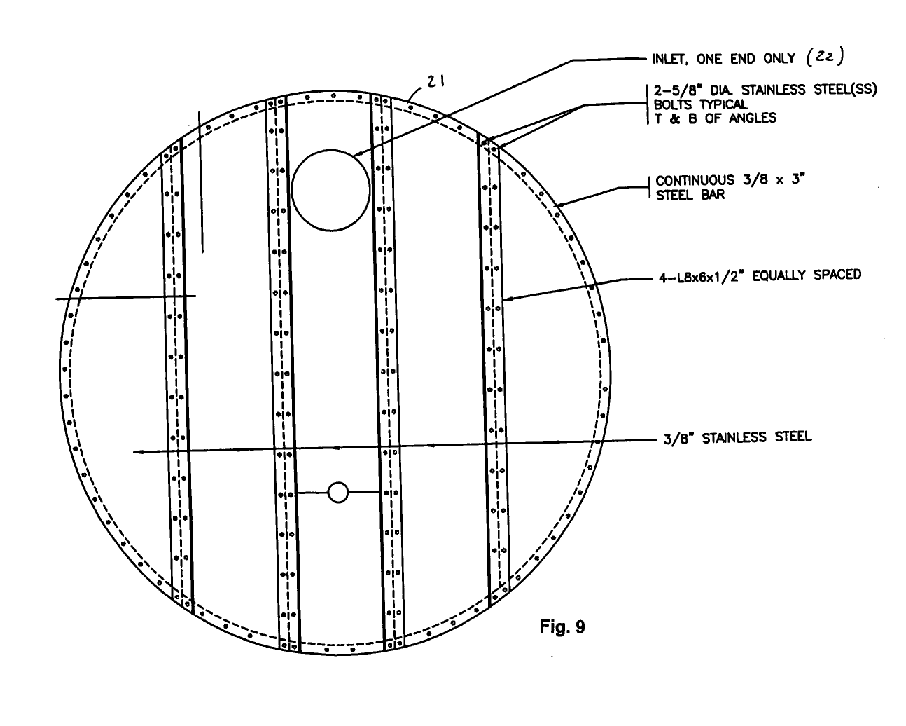

Referring to the drawings, it is seen that some of the illustrated embodiments include exemplary identification labels, dimensions and/or measurements; however, the invention and claims are not limited by or to these exemplary labels, dimensions and/or measurements which are solely provided by way of example and not limitation. In the drawings, like reference characters designate like or corresponding parts throughout the several views. Referring particularly to the exemplary illustrated embodiments of FIGS. 1-6 and 7-9, it is seen that in these embodiments, the invention includes a large generally cylindrical tank 21, having a horizontal orientation. A closable inlet 22 is provided at an upper location on tank 21 for receiving materials to be processed in the tank, and one or more controlled vents 24 are provided at other upper location(s) on tank 21 for removing air from the tank. One or more closable discharge openings 23 are provided at a lower location on tank 21. A suction device (not shown) is preferably attached to vent(s) 24 to affirmatively cause air to be removed from the tank.

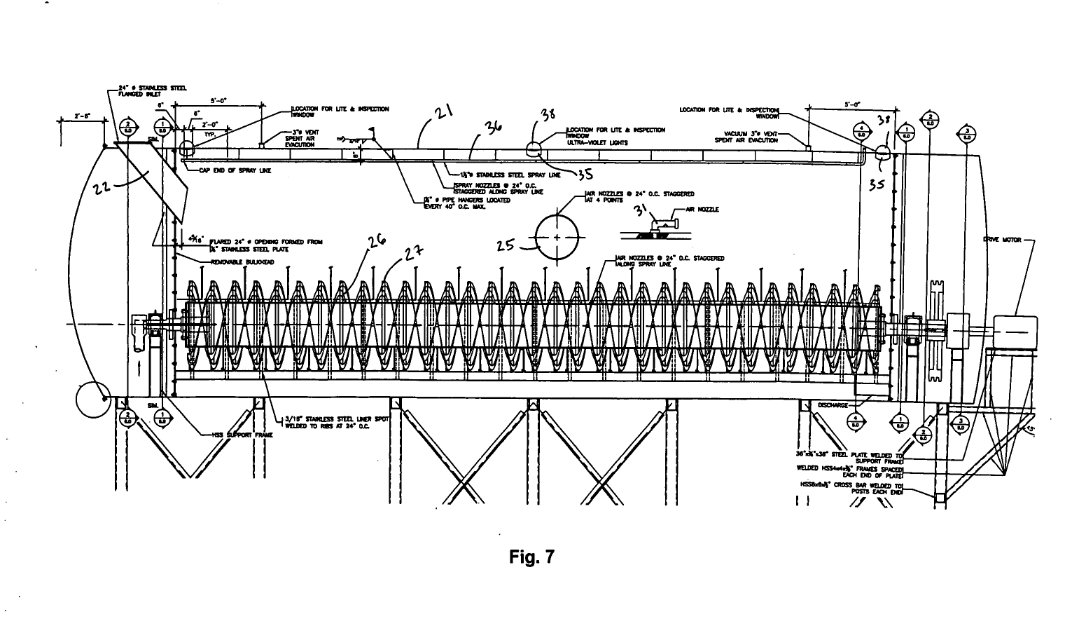

A rotatable hollow shaft 25 is provided inside tank 21 extending generally from one end of the tank to the other, as shown in FIGS. 1 and 7. The central axis of shaft 25 is preferably located below the central axis of the cylindrical tank 21 itself. Shaft 25 is provided with openings or perforations 32 along some or all of its length for introducing air into tank 21. A variable speed motor is provided on the outside of tank 21 to rotate shaft 25. An air injection system is also provided outside of tank 21 for introducing temperature controlled pressurized air into shaft 25 to force such air through the openings 32 and into the materials in the tank. A first auger 26 having a first diameter is provided on shaft 25 for urging the materials in tank 21 in one direction. A second auger 27 having a larger diameter than the first auger is also provided on shaft 25 for urging the materials in the tank 21 in the opposite direction. The combined effect of the two oppositely-urging augers 26, 27 is to agitate and mix the material in the tank when shaft 25 is rotated. The diameter of the larger auger 27 should be such that it comes close to but does not touch the bottom of tank 21. A liner or other insulation may be provided inside or outside of tank 21 to assist in maintaining a relatively constant internal temperature.

A plurality of sprayers or nozzles 30 are provided inside tank 21 in an upper area above the mixture of material, extending for some or all of the length of tank 21. These nozzles 30 are used to introduce microorganisms, enzymes, chemicals or other materials into the tank 21 to be mixed with the pulp material in the tank in order to give the resulting compost material desired characteristics. Nozzles 30 may be attached to a common line 36. Alternatively, multiple lines 36, each with its own set of nozzle(s) 30, may be provided to allow for simultaneous introduction of different materials into tank 21.

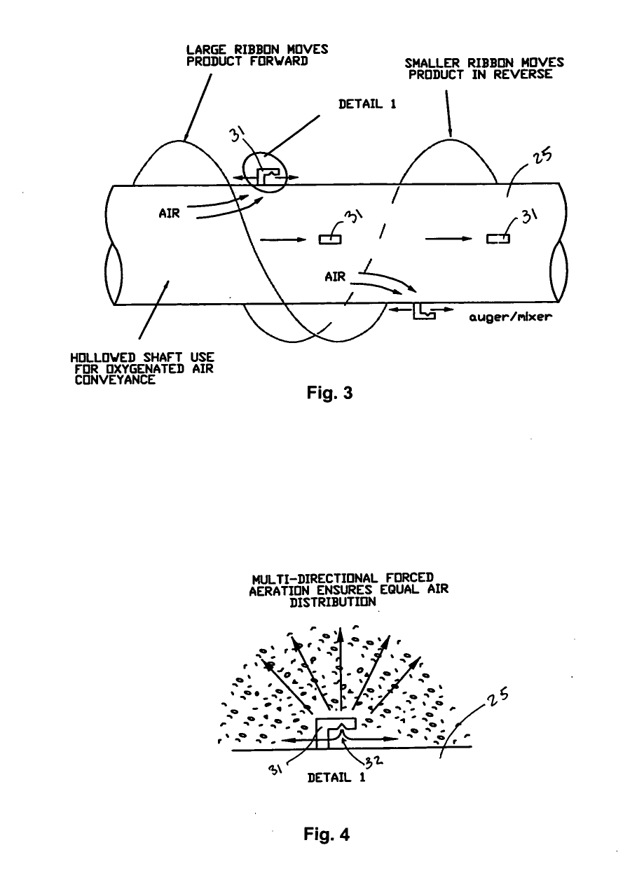

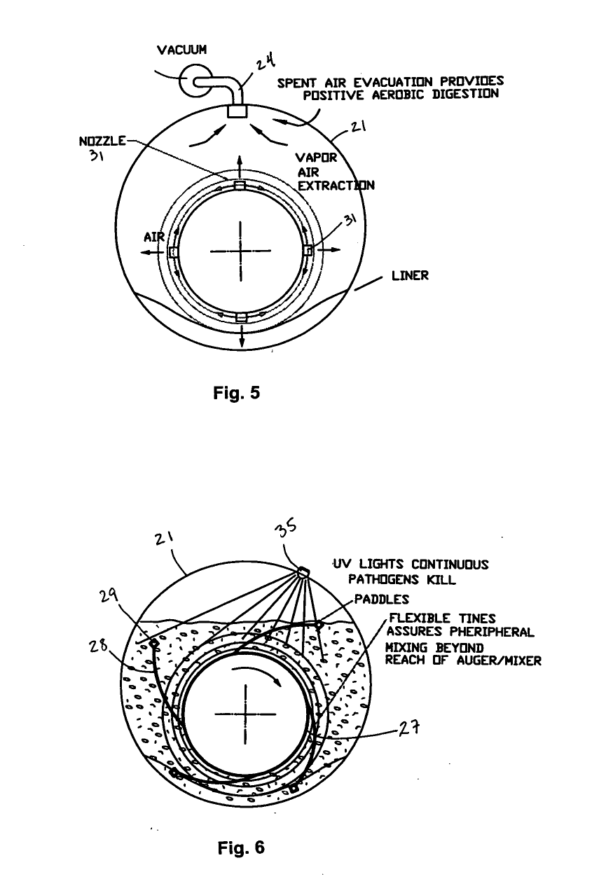

In some embodiments, the plurality of air openings 32 on shaft 25 are provided in the form of unique nozzles 31 that are spaced apart along some or all of the length of the shaft, as shown in FIGS. 3-4. In these embodiments, the nozzles 31 are uniformly spaced apart, and installed at 90 degree increments from each other in a semi-helical fashion as shown in FIG. 3. A pressurized air source causes an incoming air stream to be constantly introduced through the auger shaft 25.

In some embodiments, nozzles 31 are provided in the form of L-shaped multi-directional aeration disbursement devices at each opening to provide better air distribution into the compost pulp mix in the tank, as shown in FIG. 4. In these embodiments, a V-cut opening 32 is provided in each nozzle 31 facing the hollow shaft 25 to prevent solids from plugging the openings and to force air to be discharged in multiple directions. FIG. 5 illustrates how the air in these embodiments is disbursed around the rotating axis of shaft 25 and into the mixture in the tank.

In the embodiments illustrated in FIGS. 5-6, a plurality of flexible tines 28 are provided along the shaft 25. These tines may also include paddles or scrapers 29 provided at the ends of the tines. The tines 28 are designed to reach beyond the augers 26, 27 in order to stir the mixture in the space between the edge of the larger auger and the inside edge of the tank, and ensure that internal dead space in the tank is minimized. In other embodiments, tines 28 may be of different lengths which may or may not reach to the inside edge of tank 21. FIGS. 6 and 7 illustrate exemplary locations for ultraviolet (UV) lighting in the upper portion of the tank 21 above the mixture itself. One or more UV lights may be deployed in the upper portion of tank 21 for eradication of pathogens. Temperature sensors, level sensors, humidity sensors, pH sensors and/or sampling points (including observation and inspection windows) may also be provided in the tank, for continuous monitoring and testing of the material inside.

In use, pre-matured compost pulp is introduced into tank 21 through inlet 22, and the augers 26, 27 are activated to move and mix the material back and forth in the tank 21. If provided, the tines 28 and scrapers/paddles 29 further mix the pulp. When the level of the pulp material reaches a sufficient level to prevent drainage and allow absorption, a spray of microorganisms, enzymes or other additives is introduced through openings/nozzles 30. During or after the time this material is introduced, additional pulp is added until the tank is approximately ¾ full by volume. Pressurized air is introduced into the mixture through the nozzles/openings 31, 32 along shaft 25, and a vacuum pump, suction or other air withdrawal mechanism connected to vent(s) 24 is activated for removal of air, moisture and small particulates from the tank. The withdrawal of air should be greater than the pressurized input of air to the hollow auger shaft, preferably at about 103% of the incoming air pressure, to facilitate removal of moisture and other light particulates/contaminants in the air. It is to be appreciated that the above three steps need not necessarily be initiated simultaneously or in any particular order, but should occur at some time after the volume in the tank is sufficient to allow absorption and prevent drainage (typically about ⅓ to about ½ full by volume, or more or less).

If supplied, the ultraviolet light(s) 35 are turned on as soon as the pulp enters the tank in order to begin eradicating unwanted pathogens. If supplied, proximity and/or level sensors may be used to monitor the volume of the product in the tank. In addition, pH and thermo-couple sensors, monitors, acidity/alkalinity and temperature sensors may also be activated and monitored.

If the pulp material being introduced has come from a VCU, the incoming air should be warm and oxygenated, and preferably maintained at around 50-60° C. This helps keep the temperature of the compost mixture in the same range, around an average of 55° C. (aerobic). The mixing, oxygenation and temperature conditions should be maintained for typically about six or seven (6-7) days, in order to produce usable and relatively odor free compost. Since the pre-compost pulp material is processed in batches, it is desirable (if feasible) to provide two or more maturation tanks 21 to process different batches of pulp material at different times. It may also be desirable to remove the pulp material from a first tank when it is approximately half-way through the processing (after approximately 3-4 days), and move it to a second tank to finish processing. It is to be appreciated that a second tank may or may not need the sprayers/nozzles 30 since no additional additives may be needed during the second half of processing.

If the pulp material has not been pre-processed in a VCU, the processing time will be longer, on the order of 12-14 days. In such situations, the initial temperature will be higher (thermophilic—between 55-75° C.) for the first several days (e.g. 6-7 days), then dropping to an average of 55° C. (mesophilic—between 45-60° C.) for the remaining days (e.g. 6-7 days) of processing. In these circumstances, it may be desirable (but not necessary) to perform the thermophilic processing in a first tank at higher temperatures, and then transfer the mixture to a second tank for the longer mesophilic processing.

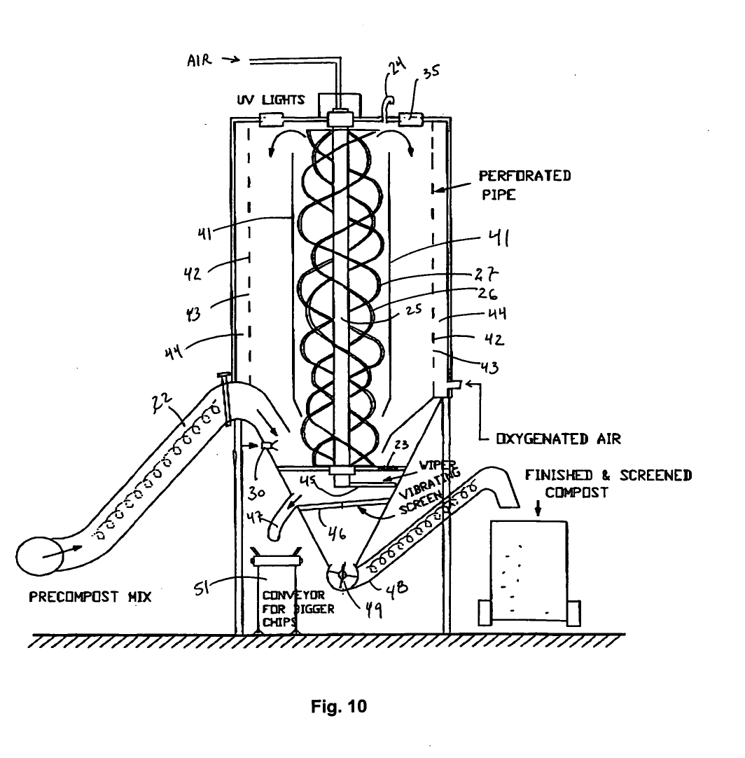

FIG. 10 illustrates an exemplary alternative vertical embodiment of the present invention. In the vertical embodiments, the lower portion of tank 21 has a conical shape, and the pre-compost pulp mixture is introduced into the tank near the bottom at a closable inlet 22. Hollow shaft 25 has a generally vertical orientation, and is provided with openings 32 along its length for the introduction of warm air. Nozzles 31 (as illustrated in FIGS. 3 and 4) may also be provided on the shaft. Shaft 25 includes two augers 26, 27. One auger 27 urges the mixture in the tank 21 in an upward direction, and the other auger 26 urges the mixture in a downward direction. The augers 26, 27 and shaft 25 are surrounded by an interior cylindrical wall 41 that has a diameter slightly larger than the outside diameter of the larger auger 27. Wall 41 does not reach the top or the bottom of tank 21, allowing space at both ends for the mixture in the tank to enter and exit. The lower portion of wall 41 is preferably slightly conical. The larger/stronger auger 27 causes the mixture as a whole to move in a generally vertical direction inside wall 41, as illustrated by the arrows in FIG. 10.

Another cylindrical wall 42 is provided along the inside edge of tank 21, defining an annular air space or plenum 44 between wall 42 and the outside wall of tank 21. In addition to introducing air through perforations in the auger shaft 25, in the vertical embodiments warm pressurized air is also forced into the annular space 44 between wall 42 and tank 21. Perforations or openings 43 are provided in wall 42 to allow this air to mingle with the mixture in the tank 21 as it descends in the area between cylindrical walls 41 and 42. Openings 43 may be provided in uniform or random patterns along wall. 42, and/or may be provided in the form illustrated in FIGS. 3-4.

One or more vents 24 are provided at the top of tank 21 for removing air. Vacuum or suction units (not shown) may be attached to the vent(s) 42 to remove such air for filtering. One or more ultraviolet lights 35 may be provided at the top of the tank, and may be placed in a uniform or random pattern. A sprayer or nozzle 30 is provided near the inlet 22 at the bottom of the tank for introducing microorganisms, enzymes or other, materials to be added to the mixture in the tank.

It is to be appreciated that in the vertical embodiments, the pulp enters at the bottom of the tank 21, where it is mixed with additives through input 30. The mixture is then moved upward inside pipe 41 by the action of the augers. The mixture then spills over the top of pipe 41, and moves downward in the space between pipes 41 and 42 until it reaches the bottom of tank 21 where the augers move it back upward again in a continuous cycle. This circulating motion causes the mixture in the tank to mix with any microorganisms, enzymes or other materials introduced through sprayer/nozzle 30, to mix with the air/oxygen pumped in through shaft openings 32 and plenum openings 43, and be exposed to the ultraviolet light from lighting unit(s) 35.

A closable gate or discharge 23 is provided in a bottom wall of tank 21 for releasing matured compost from the tank 21. When discharge 23 is opened, a rotating member (wiper) 45 is used to stir the matured compost over a screen 46. Larger chips of compost that do not pass through the screen (e.g. larger than 2″) are knocked out of the way by wiper 45 and dropped into a conveyor 51. The remaining compost material passes through screen 46 and is moved through a discharge conveyor 48 by rotating valve unit 49. In some embodiments, screen 46 may be provided with a vibration mechanism, to accelerate its screening/separation function. It is to be appreciated that screen 46 may be of any suitable size/mesh in order to produce compost of a desired fineness.

In use, pre-matured compost pulp is introduced into vertical tank 21 through lower inlet 22, and the augers 26, 27 are activated to mix the material and move it upward inside pipe 41. Shortly after the pulp material begins introduction into the tank, microorganisms, enzymes or other additives are introduced through nozzle 30. This material is introduced with or after additional pulp until the tank is approximately ¾ full by volume. Meanwhile, pressurized warm air is introduced into the mixture through the shaft openings 32 (and nozzles 31 if provided) and the openings 43 in wall 42. At the same time, a vacuum pump or other withdrawal mechanism connected to vent(s) 24 is activated for removal of air and moisture from the tank. The withdrawal of air should be greater than the pressurized input of air to the annular space to help remove moisture and light particulates. If supplied, the ultraviolet light(s) 35 are turned on as soon as the pulp enters the tank in order to begin eradicating unwanted pathogens. If supplied, proximity and/or level sensors may be used to monitor the volume of the product in the tank. In addition, pH and thermo-couple sensors, monitors, acidity/alkalinity and temperature sensors may also be activated and monitored.

As with the horizontally oriented embodiments of the invention, if the pulp material being introduced has come from a VCU, the incoming air should be warm and oxygenated, and preferably maintained at around 50-60° C. This helps keep the temperature of the compost mixture in the same range, around an average of 55° C. (aerobic). The mixing, oxygenation and temperature conditions should be maintained for typically about six or seven (6-7) days, in order to produce usable and relatively odor free compost. Since the pre-compost pulp material is processed in batches, it is desirable (if feasible) to provide two or more maturation tanks 21 to process different batches of pulp material at different times. It may also be desirable to remove the pulp material from a first tank when it is approximately half-way through the processing (after approximately 3-4 days), and move it to a second tank to finish processing. It is to be appreciated that a second tank may or may not need input 30 since no additional additives may be needed during the second half of processing.

As with the horizontally oriented embodiments of the invention, if the pulp material has not been pre-processed in a VCU, the processing time will be longer, on the order of 12-14 days. In such situations, the initial temperature will be higher (thermophilic—between 55-75° C.) for the first several days (e.g. 6-7 days), then dropping to an average of 55° C. (mesophilic—between 45-60° C.) for the remaining days (e.g. 6-7 days) of processing. In these circumstances, it may be desirable (but not necessary) to perform the thermophilic processing in a first tank at higher temperatures, and then transfer the mixture to a second tank for the longer mesophilic processing.

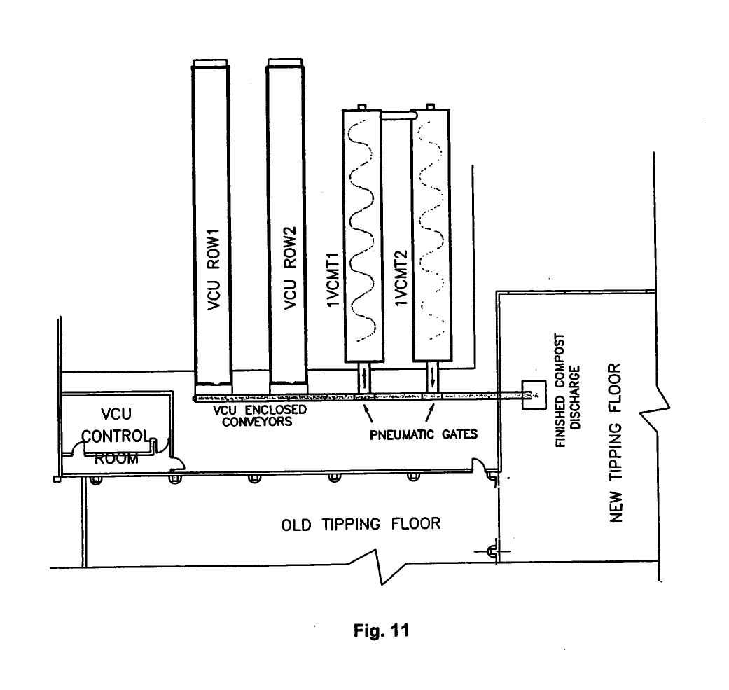

FIG. 11 provides an illustration of a typical layout for an implementation of vertically oriented embodiments of the present invention.

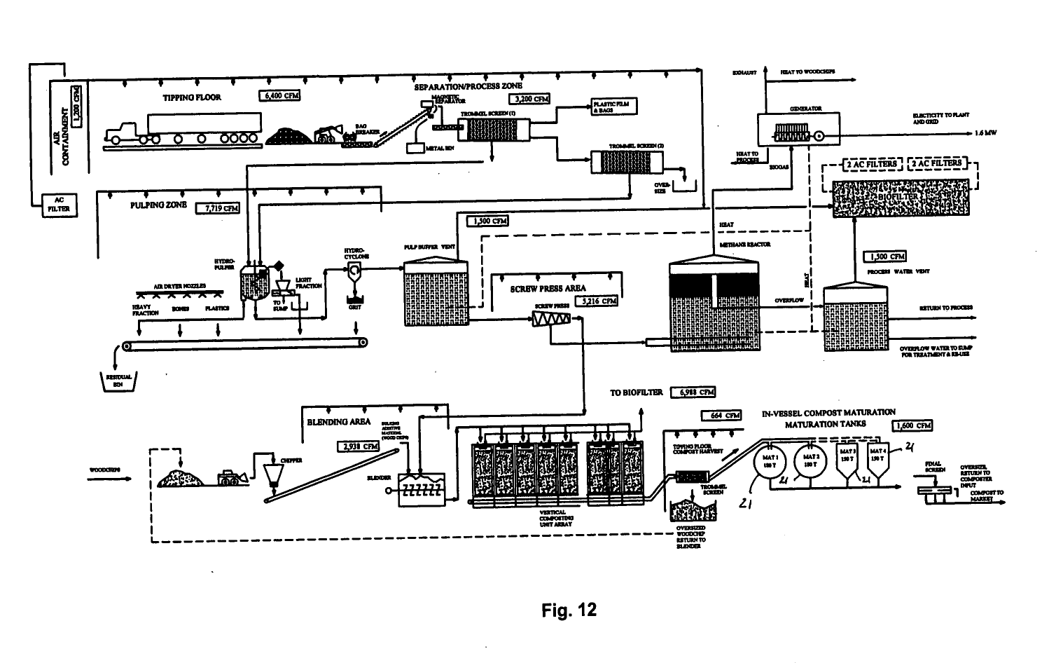

FIG. 12 provides an illustration of an implementation of the present invention in an existing composting system, receiving the output from a set of VCUs. A pair of horizontally oriented embodiments of the maturation tanks 21 of the present invention are shown in this illustrated exemplary embodiment (MAT 1 and MAT 2), allowing processing of two batches of pre-compost pulp simultaneously. It is to be appreciated that additional tanks, such as the illustrated proposed vertically oriented tanks 21 (MAT 3 and MAT 4) may be used with or as alternatives to the horizontally oriented embodiments.

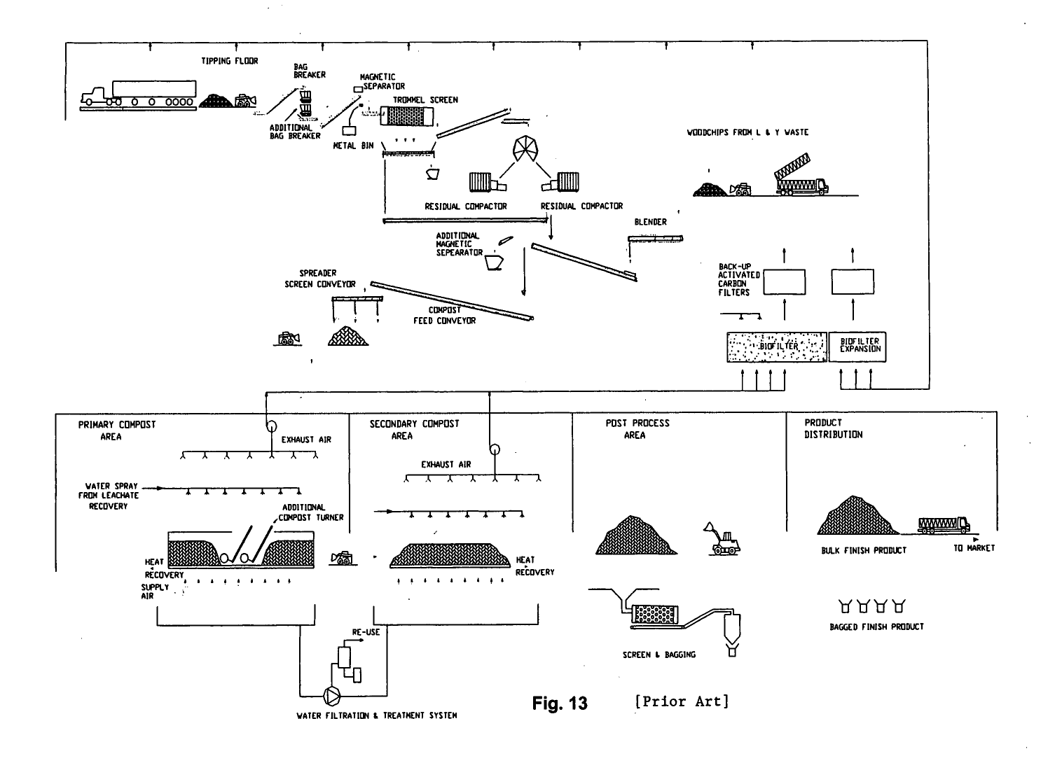

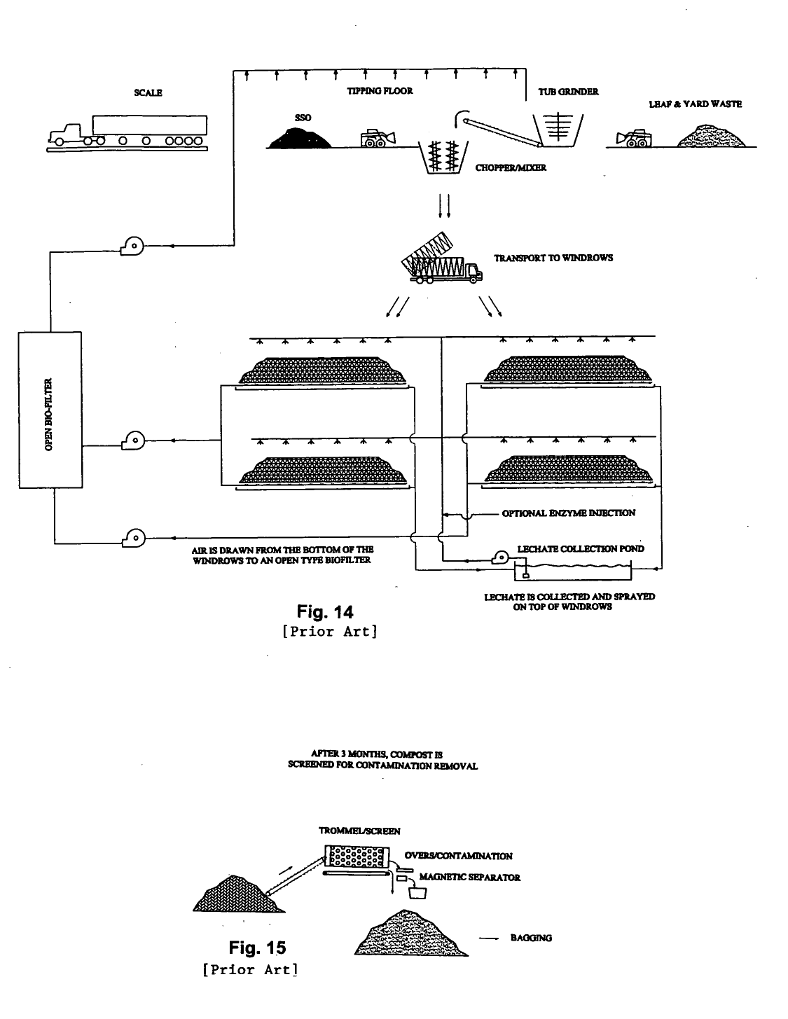

FIG. 13 illustrates a typical prior art in-tunnel (aerobic) composting system, and FIG. 14 illustrates a typical prior art windrow (aerobic) composting system. FIG. 15 illustrates screening of the final compost product (e.g. removal of residual product larger than ⅜″ in spherical size).

In some embodiments, multiple tanks of the present invention may be utilized in series or in parallel. It is to be appreciated that in some embodiments, one or more horizontally oriented tanks of the present invention may be used in parallel with each other or in parallel with one or more vertically oriented tanks of the present invention. In other embodiments, one or more horizontally oriented tanks of the present invention may be used in series with each other or in series with one or more vertically oriented tanks of the present invention. Multiple tank embodiments are particularly suited for use with pre-compost pulp that has not been pre-processed by a VCU, since different processing temperatures are used at different times.

It is to be understood that variations and modifications of the present invention may be made without departing from the scope thereof. It is also to be understood that the present invention is not to be limited by the specific embodiments disclosed herein, but only in accordance with the appended claims when read in light of the foregoing specification.

What is claimed is:

1. An apparatus for use in composting comprising:
a. an elongated generally horizontally oriented maturation tank having a central axis, a closable inlet opening, a closable outlet opening, and at least one vent;
b. a hollow rotatable shaft having a plurality of openings therein deployed in said tank;
c. a first auger attached to said shaft for urging materials in said tank in a first direction along said shaft;
d. a second auger attached to said shaft for urging materials in said tank in an opposite direction along said shaft;
e. a plurality of flexible tines attached to said shaft;
f. an input for introducing temperature controlled forced air into said hollow shaft;
g. a suction in communication with said at least one vent for removing air from said tank at a greater strength than said forced air input;
h. at least one fluid input located in an upper region inside said tank; and
i. at least one ultraviolet light source located in an upper region inside said tank.

2. The apparatus of claim 1 wherein said shaft has an axis and is disposed inside said tank such that the axis of said shaft is offset from the axis of said tank.

3. The apparatus of claim 1 wherein at least one scraper is attached at an end of at least one of said tines.

4. The apparatus of claim 1 wherein said first auger has a first diameter, and said second auger has a diameter that is smaller than said first diameter.

5. The apparatus of claim 1 wherein a plurality of nozzles are attached to said plurality of openings on said shaft, each such nozzle having an L-shaped cross section and a V-cut opening therein facing said shaft.

6. The apparatus of claim 1 wherein said plurality of openings on said shaft are uniformly spaced apart, and installed in a semi-helical fashion.

7. The apparatus of claim 6 wherein a plurality of nozzles are attached to said plurality of openings on said shaft, each such nozzle having an L-shaped cross section and a V-cut opening therein facing said shaft.

8. The apparatus of claim 6 wherein said plurality of openings on said shaft are provided at 90 degree increments from each other.

9. An apparatus for use in composting comprising:
a. a maturation tank having a central axis, a closable inlet opening, a closable outlet opening, and at least one vent;
b. a hollow rotatable shaft in said tank having a plurality of openings therein;
c. a first auger attached to said shaft for urging materials in said tank in a first direction along said shaft;
d. a second auger attached to said shaft for urging materials in said tank in an opposite direction along said shaft;
e. an input for introducing forced air into said hollow shaft; and
f. at least one fluid input to the inside of said tank.

10. The apparatus of claim 9 wherein a plurality of nozzles are attached to said plurality of openings on said shaft.

11. The apparatus of claim 10 wherein each of said nozzles has an L-shaped cross section and a V-cut opening therein facing said shaft.

12. The apparatus of claim 9 wherein a plurality of flexible tines are attached to said shaft.

13. The apparatus of claim 12 wherein at least one scraper is attached at an end of at least one of said tines.

14. The apparatus of claim 9 wherein at least one ultraviolet light source is provided in an upper region inside said tank.

15. The apparatus of claim 9 wherein a suction is provided in communication with said at least one vent for removing air from said tank.

16. The apparatus of claim 9 wherein said plurality of openings on said shaft are uniformly spaced apart, and installed in a semi-helical fashion.

17. The apparatus of claim 16 wherein a plurality of nozzles are attached to said plurality of openings on said shaft, each such nozzle having an L-shaped cross section and a V-cut opening therein facing said shaft.

18. The apparatus of claim 17 wherein said plurality of openings on said shaft are provided at 90 degree increments from each other.

19. An apparatus for use in composting comprising:
a. an elongated generally vertically oriented maturation tank having a generally cylindrical exterior wall, a semi-conical lower portion, a central axis, a closable inlet opening located at a lower region thereof, a floor located in a lower region of said tank having a closable opening thereon, an outlet located below said floor, and at least one upper vent;
b. a hollow rotatable shaft having a plurality of openings therein deployed in said tank along said axis;
c. a first auger attached to said shaft for urging materials in said tank in an upward direction along said shaft;
d. a second auger attached to said shaft for urging materials in said tank in a downward direction along said shaft;
e. a first hollow open ended tubular member deployed in said tank around said rotatable shaft and augers;
f. a second larger hollow tubular member deployed in said tank adjacent to said cylindrical wall defining an annular plenum between said wall and said second tubular member, said second tubular member having a plurality of openings located therein; and
g. at least one input for introducing forced air into said hollow shaft and said plenum.

20. The apparatus of claim 19 wherein at least one ultraviolet light source is provided in an upper region inside said tank.

21. The apparatus of claim 19 wherein said first auger has an outer diameter that is slightly smaller than the inside diameter of said first tubular member, and said second auger has a diameter that is smaller than that of said first auger.

22. The apparatus of claim 19 wherein at least one fluid input is provided in a lower region inside said tank.

23. The apparatus of claim 19 wherein a rotatable wiper is provided below said floor.

24. The apparatus of claim 19 wherein a screen is provided between said floor and said outlet.

25. The apparatus of claim 24 wherein a second outlet is provided above said screen.

26. The apparatus of claim 24 wherein insulation is provided for said tank.

27. A system for generating compost comprising at least two maturation tanks and at least one fluid communication channel between such tanks, each such tank further comprising:
a. an exterior wall having a closable inlet opening, a closable outlet opening, and at least one vent;
b. a hollow rotatable shaft in such tank having a plurality of openings therein;
c. a first auger attached to each such shaft for urging materials in such tank in a first direction along such shaft;
d. a second auger attached to each such shaft for urging materials in such tank in an opposite direction along such shaft;
e. an input for introducing forced air into each such hollow shaft; and
f. at least one fluid input to the inside of each such tank.

28. A system for generating compost comprising:
a. a first maturation tank having a closable inlet opening, a closable outlet opening, and at least one vent;
b. a hollow rotatable shaft in said first tank having a plurality of openings therein;
c. a first auger attached to said shaft for urging materials in said first tank in a first direction along said shaft;
d. a second auger attached to said shaft for urging materials in said first tank in an opposite direction along said shaft;
e. an input for introducing forced air into said hollow shaft;
f. at least one fluid input to the inside of said first tank;
g. a second maturation tank having a generally cylindrical vertically oriented exterior wall, a semi-conical lower portion, a central axis, a closable inlet opening located at a lower region thereof, a floor located in a lower region of said second tank having a closable opening thereon, an outlet located below said floor, and at least one upper vent;
h. a second hollow rotatable shaft having a plurality of openings therein deployed in said second tank along said axis;
i. a third auger attached to said second shaft for urging materials in said second tank in an upward direction along said second shaft;
j. a fourth auger attached to said second shaft for urging materials in said second tank in a downward direction along said second shaft;
k. a first hollow open ended tubular member deployed in said second tank around said second rotatable shaft and augers;
l. a second larger hollow tubular member deployed in said second tank adjacent to said cylindrical wall defining an annular plenum between said wall and said second tubular member, said second tubular member having a plurality of openings located therein; and
g. at least one input for introducing forced air into said second hollow shaft and said plenum.

29. An apparatus for use in composting comprising:
a. a tank means having an inlet means, an outlet means, and at least one vent means;
b. a hollow rotatable means in said tank means having a plurality of openings therein;
c. a first means attached to said rotatable means for urging materials in said tank in a first direction;
d. a second means attached to said rotatable means for urging materials in said tank in an opposite direction;
e. an means for introducing forced air into said rotatable means; and
f. at least one means for introducing fluid to the inside of said tank.

30. A method for generating compost comprising the steps of:
a. introducing a quantity of organic pulp into a mixing tank;
b. rotating a hollow shaft in said tank, said shaft having a plurality of openings therein and said shaft having dual direction augers located thereon;
c. introducing a pre-determined quantity of an additive to the pulp to form a mixture, said additive comprising a member selected from the group of: at least one microorganism, at least one enzyme, and combinations thereof;
d. introducing pressurized air into said mixture through the openings in said shaft;
e. activating a suction to remove air from said tank; and
f. continuing the rotation of said shaft, the introduction of pressurized air, and the suction of air for a time period of between about 1 and about 6 days.

31. The method of claim 30 comprising the additional step of maintaining the temperature of said mixture between about 45° C. and about 60° C.

32. The method of claim 30 wherein the temperature of said mixture is maintained at an average of about 55° C.

33. The method of claim 30 wherein said time period is about 3 days.

34. The method of claim 30 comprising the additional step of activating at least one ultraviolet light source inside said tank.

35. The method of claim 30 wherein said additive makes up no more than one percent of said mixture.

36. The method of claim 30 wherein the air removed by said suction is oxygenated and reintroduced into said mixture.

37. The method of claim 30 wherein air is removed by said suction at a greater strength than the pressurized air introduced into said mixture.

38. The method of claim 30 wherein a plurality of nozzles are attached to said plurality of openings on said shaft, each such nozzle having an L-shaped cross section and a V-cut opening therein facing said shaft.

39. The method of claim 30 wherein said tank has a generally cylindrical shape and a generally horizontal orientation.

40. The method of claim 30 wherein said tank has a generally cylindrical wall and a generally vertical orientation, an open-ended tubular member is provided around said augers, and wherein said mixture flows in an upward direction inside said tubular member adjacent to said augers and in a downward direction between said tubular member and said cylindrical wall.

41. The method of claim 40 wherein a second hollow tubular member is provided in said tank adjacent to said cylindrical wall defining an annular plenum between said wall and said second tubular member and also defining a space between said first and second tubular members for the downward flow of said mixture, said second tubular member having a plurality of openings located therein, and comprising the additional step of introducing pressurized air into said mixture through the plurality of openings in said second tubular member.

42. A method for generating compost comprising the steps of:
a. introducing a quantity of organic pulp into a mixing tank;
b. rotating a hollow shaft in said tank, said shaft having a plurality of openings therein and said shaft having dual direction augers located thereon;
c. introducing a predetermined quantity of an additive to the pulp to form a mixture, said additive comprising a member selected from the group of: at least one microorganism, at least one enzyme, and combinations thereof;
d. introducing pressurized air into said mixture through the openings in said shaft;
e. activating a suction to remove air from said tank; and
f. maintaining the temperature of said mixture at between about 55° C. and about 75° C. for a first time period of between about 6 and about 7 days while continuing the rotation of said shaft, the introduction of pressurized air, and the suction of air; and
g. maintaining the temperature of said mixture at between about 45° C. and about 60° C. for a second time period of between about 6 and about 7 days while continuing the rotation of said shaft, the introduction of pressurized air, and the suction of air.

43. The method of claim 42 comprising the additional step of activating at least one ultraviolet light source inside said tank.

43. The method of claim 42 wherein said additive makes up no more than one percent of said mixture.

44. The method of claim 42 wherein the air removed by said suction is oxygenated and reintroduced into said mixture.

46. The method of claim 42 wherein air is removed by said suction at a greater strength than the pressurized air introduced into said mixture

47. The method of claim 42 wherein a plurality of nozzles are attached to said plurality of openings on said shaft, each such nozzle having an L-shaped cross section and a V-cut opening therein facing said shaft.

48. The method of claim 42 wherein said tank has a generally cylindrical shape and a generally horizontal orientation.

49. The method of claim 42 wherein said tank has a generally cylindrical wall and a generally vertical orientation, an open-ended tubular member is provided around said augers, and wherein said mixture flows in an upward direction inside said tubular member adjacent to said augers and in a downward direction between said tubular member and said cylindrical wall.

50. The method of claim 49 wherein a second hollow tubular member is provided in said tank adjacent to said cylindrical wall defining an annular plenum between said wall and said second tubular member and also defining a space between said first and second tubular members for the downward flow of said mixture, said second tubular member having a plurality of openings located therein, and comprising the additional step of introducing pressurized air into said mixture through the plurality of openings in said second tubular member.

51. A method for generating compost comprising the steps of:
a. introducing a quantity of organic pulp into a first mixing tank;
b. rotating a hollow shaft in said tank, said shaft having a plurality of openings therein and said shaft having dual direction augers located thereon;
c. introducing a predetermined quantity of an additive to the pulp to form a mixture, said additive comprising a member selected from the group of: at least one microorganism, at least one enzyme, and combinations thereof;
d. introducing pressurized air into said mixture through the openings in said shaft;
e. activating a suction to remove air from said tank;
f. maintaining a first temperature for said mixture for a first period, of time while continuing the rotation of said shaft, the introduction of pressurized air, and the suction of air;
g. transferring said mixture to a second tank;
h. rotating a second hollow shaft in said second tank, said second shaft having a plurality of openings therein and said second shaft having dual direction augers located thereon;
i. introducing pressurized air into said mixture through the openings in said second shaft;
e. activating a second suction to remove air from said second tank; and
f. maintaining a second temperature for said mixture for a second period of time while continuing the rotation of said second shaft, the introduction of pressurized air, and the suction of air.

52. The method of claim 51 wherein said first temperature is between about 45° C. and about 60° C., said first period of time is between about 1 and about 3 days, said second temperature is between about 45° C. and about 60° C., and said second period of time is between about 1 and about 3 days.

53. The method of claim 51 wherein said first temperature is between about 55° C. and about 75° C., said first period of time is between about 6 and about 7 days, said second temperature is between about 45° C. and about 60° C., and said second period of time is between about 6 and about 7 days.

54. The method of claim 51 wherein the temperature of said mixture is maintained at an average of about 55° C. in both tanks.
